(19) United States
(12) Patent Application Publication
(10) Pub. No.: US 2022/0369947 A1
(43) Pub. Date: Nov. 24, 2022

(54) ESTIMATION OF DISTALLY-LOCATED MULTIPORT NETWORK PARAMETERS USING MULTIPLE TWO-WIRE PROXIMAL MEASUREMENTS

(71) Applicant: Analog Devices International Unlimited Company, Limerick (IE)

(72) Inventors: Goutam DUTTA, Bengaluru (IN); Sriram GANESAN, Bangalore (IN); Venugopal GOPINATHAN, Boston, MA (US)

(73) Assignee: Analog Devices International Unlimited Company, Limerick (IE)

(21) Appl. No.: 17/748,181

(22) Filed: May 19, 2022

Related U.S. Application Data
(60) Provisional application No. 63/190,855, filed on May 20, 2021.

(51) Int. Cl. A61B 5/053; G01R 27/16

(52) U.S. Cl. CPC A61B 5/053; G01R 27/16

(57) ABSTRACT

Accurately measuring bio-impedance is important for sensing properties of the body. Unfortunately, contact impedances can significantly degrade the accuracy of bio-impedance measurements. To address this issue, a method is provided for estimating an impedance matrix of parasitic network disposed between a first network and a second network of a bio-impedance measurement system, the method comprising determining an impedance matrix for the first network (Z_{MUX}) based on an impedance matrix for the second network (Z_{LOAD}) for at least one known load condition; fitting Z_{MUX} values for Z_{LOAD} for the at least one known load condition to estimate parameters of the impedance matrix of the intervening network.

### CROSS-REFERENCE TO RELATED APPLICATION

This application claims the benefit of and priority to U.S. Patent Application Ser. No. 63/190,855, filed May 20, 2021, entitled “TECHNIQUE FOR ACCURATE ESTIMATION OF MULTIPORT NETWORK PARAMETERS LOCATED DISTALLY USING MULTIPLE TWO-WIRE PROXIMAL MEASUREMENTS,” which is incorporated herein by reference in its entirety.

### TECHNICAL FIELD OF THE DISCLOSURE

The present invention relates to the field of integrated circuits, in particular to bio-impedance measurements.

### BACKGROUND

Impedance measurements of the body, referred herein as bio-impedance measurements, have many applications in healthcare and consumer applications. Bio-impedance measurements can be made by electrodes provided in body-worn systems, or wearable devices, such as wrist watches, chest bands, head bands, patches, and so on. Circuitry coupled to the electrodes can derive the unknown impedance of the body on which the electrodes are placed. Impedance measurements can be particularly useful for vital-signs monitoring, sensing of tissues and fluid level in the body for purposes of detecting signs of pulmonary edema, or assess body composition. Moreover, electrical impedance tomography is an emerging non-invasive technique of medical imaging. Due to various challenges, making an accurate bio-impedance measurement is not trivial.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 illustrates a system having electrodes and circuitry for performing one exemplary way of making an impedance measurement of bio-impedance, according to some embodiments of the disclosure;

FIG. 2 illustrates input capacitances present in circuitry that performs an impedance measurement of bio-impedance, according to some embodiments of the disclosure;

FIG. 3 illustrates current leakage present in circuitry that performs an impedance measurement of bio-impedance, according to some embodiments of the disclosure;

FIGS. 4A and 4B illustrate simplified block diagrams of a method for measuring a simple two-wire impedance with a non-ideal meter;

FIG. 5 illustrates representation of a body network may be represented as a 3×3 impedance matrix of a 3-port network;

FIGS. 6-11 illustrate example methods of measurement calibration, according to some embodiments of the disclosure;

FIGS. 12A-12D illustrate a mapping of an impedance matrix from mux-to-load end, according to embodiments of the disclosure;

FIGS. 13 and 14A-14H illustrate calibration operations according to some embodiments of the disclosure; and

FIGS. 15 and 16 illustrate de-embedding operations according to some embodiments of the disclosure; and

FIG. 17 is a block diagram illustrating an example system that may be configured for implementing one or more aspects of embodiments described herein.

### DESCRIPTION OF EXAMPLE EMBODIMENTS OF THE DISCLOSURE

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C). The term “between,” when used with reference to measurement ranges, is inclusive of the ends of the measurement ranges. When used herein, the notation “A/B/C” means (A), (B), and/or (C).

The description uses the phrases “in an embodiment” or “in embodiments,” which may each refer to one or more of the same or different embodiments. Furthermore, the terms “comprising,” “including,” “having,” and the like, as used with respect to embodiments of the present disclosure, are synonymous. The disclosure may use perspective-based descriptions such as “above,” “below,” “top,” “bottom,” and “side”; such descriptions are used to facilitate the discussion and are not intended to restrict the application of disclosed embodiments. The accompanying drawings are not necessarily drawn to scale. Unless otherwise specified, the use of the ordinal adjectives “first,” “second,” and “third,” etc., to describe a common object, merely indicate that different instances of like objects are being referred to and are not intended to imply that the objects so described must be in a given sequence, either temporally, spatially, in ranking or in any other manner.

In the following detailed description, reference is made to the accompanying drawings that form a part hereof, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

The following disclosure describes various illustrative embodiments and examples for implementing the features and functionality of the present disclosure. While particular components, arrangements, and/or features are described below in connection with various example embodiments, these are merely examples used to simplify the present disclosure and are not intended to be limiting. It will of course be appreciated that in the development of any actual embodiment, numerous implementation-specific decisions must be made to achieve the developer's specific goals, including compliance with system, business, and/or legal constraints, which may vary from one implementation to another. Moreover, it will be appreciated that, while such a development effort might be complex and time-consuming; it would nevertheless be a routine undertaking for those of ordinary skill in the art having the benefit of this disclosure.

In the specification, reference may be made to the spatial relationships between various components and to the spatial orientation of various aspects of components as depicted in the attached drawings. However, as will be recognized by those skilled in the art after a complete reading of the present disclosure, the devices, components, members, apparatuses, etc. described herein may be positioned in any desired orientation. Accordingly, the use of terms such as “above”, “below”, “upper”, “lower”, “top”, “bottom”, or other similar terms to describe a spatial relationship between various components or to describe the spatial orientation of aspects of such components, should be understood to describe a relative relationship between the components or a spatial orientation of aspects of such components, respectively, as the components described herein may be oriented in any desired direction. When used to describe a range of dimensions or other characteristics (e.g., time, pressure, temperature, length, width, etc.) of an element, operations, and/or conditions, the phrase “between X and Y” represents a range that includes X and Y.

Further, the present disclosure may repeat reference numerals and/or letters in the various examples. This repetition is for the purpose of simplicity and clarity and does not in itself dictate a relationship between the various embodiments and/or configurations discussed. Example embodiments that may be used to implement the features and functionality of this disclosure will now be described with more particular reference to the accompanying FIGURES.

Overview

Measuring bio-impedance can be particularly useful for measuring body impedance for detecting fluid level of the lungs or measuring thoracic impedance. Measuring bio-impedance can also be useful in electrical impedance tomography to determine a composition of the body (e.g., imaging of tissues and bones) in a non-invasive manner by making bio-impedance measurements at different frequencies. Measuring bio-impedance can be useful in measuring respiration activity, where respiration activity can be obtained by observing variation in thorax impedance. Measuring bio-impedance and the contact impedances means that respiration activity can be obtained even in the presence of motion, since variations in contact impedances can be taken into account. Users such as athletes and patients can greatly benefit from such applications.

For a variety of reasons, the impedances measurement schemes described herein can be used in a variety of situations. For instance, the impedances measurement scheme can be used to, non-invasively, obtain the body's composition, determine thoracic impedance, determine respiration activity in the presence of motion, etc.

Bio-impedance measurements of a body, or portions thereof, of a subject can be utilized for determining health characteristics (such as heart conditions) of the subject. However, performing bio-impedance measurements may present sources of error that result in the bio-impedance measurement being imprecise. For four-way or four-wire impedance measurement approaches of measuring the bio-impedance, the measurement system may present a parasitic network that can cause the measured bio-impedance to be imprecise.

Estimation of network parameters located distally through proximal measurements are negatively impacted by the effects of an intervening parasitic network (or simply an “intervening network”). A multiport network located distally would traditionally require a multiport measurement entity. Systems described herein provide accurate methods for estimating a distally-located multiport network through multiple measurements made proximally using different configurations. The intervening parasitic multiport network is accurately estimated during a calibration sequence and the effects thereof are de-embedded or calibrated out. This result is a precise estimation of the distal network.

In general, embodiments described herein include a model of a generalized intervening parasitic network. Six different two-wire measurements are performed to construct the complete Z-matrix of the network as seen from a multiplexer (mux). Measurements are calibrated with respect to RCAL. Measurements are taken on known loads in the factory to estimate the intervening parasitic network. The parasitic network is calibrated and calibration parameters are stored in flash memory. The calibration parameters are later used to de-embed any loads to estimate the load impedance matrix.

Impedance Measurement

One technique for accurate impedance measurement nullifying the effects of contact impedance is a four-terminal sensing scheme, or four-wire impedance measurement scheme. Sometimes it is referred to as Kelvin sensing. The technique involves using four electrodes placed on the body to sense or derive an unknown bio-impedance. In particular, the bio-impedance may comprise an impedance presented by the body, or portion thereof, of the subject to the flow of current that may be applied to the body. The technique may involve having a plurality of electrodes placed on a body of the subject. For example, the technique may involve having four electrodes placed on a body of the subject and the electrodes may be utilized to sense or derive an unknown bio-impedance of a portion of the body of the subject corresponding to the placement of the electrodes.

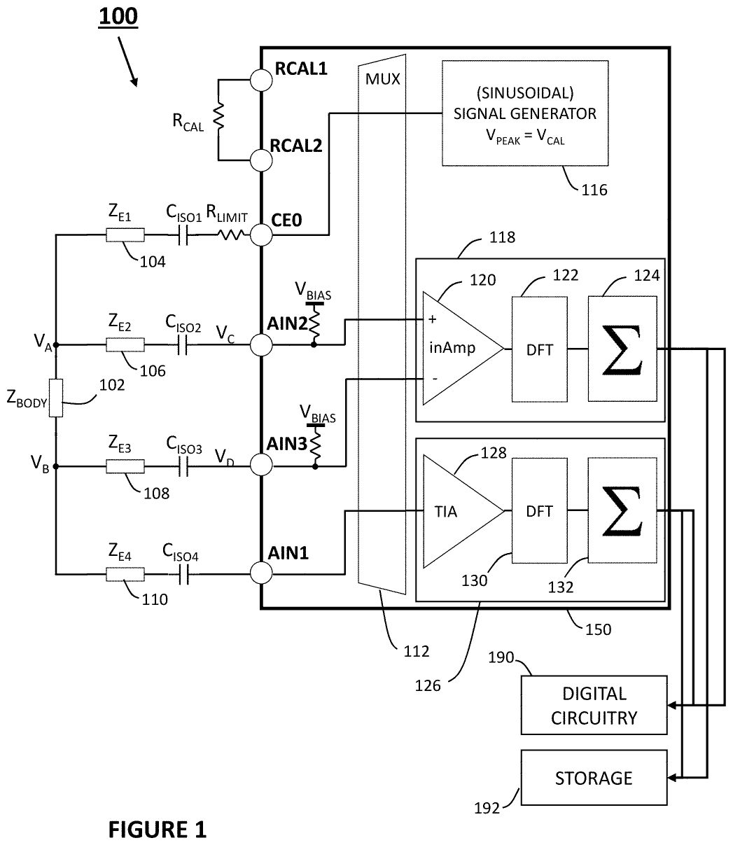

FIG. 1 illustrates a system 100 having electrodes and circuitry for performing one exemplary way making a four-wire impedance measurement of bio-impedance, according to some embodiments of the disclosure. In the FIGURE, the unknown bio-impedance is shown as Z_{BODY}. The system 100 includes electrodes 104, 106, 108, and 110 (or contacts to the body). The electrodes 104, 106, 108, and 110 have respective contact impedances Z_{E1}, Z_{E2}, Z_{E3}, and Z_{E4}. Contact impedances Z_{E1}, Z_{E2}, Z_{E3}, and Z_{E4} can represent skin-electrode impedance of the electrodes 104, 106, 108, and 110, respectively. Circuitry 150, packaged as an integrated circuit or chip, has pins (or connections) to which the electrodes are connected. Pin CE0 is electrically coupled to electrode 104. Pin AIN2 is electrically coupled to electrode 106. Pin AIN3 is electrically coupled to electrode 108. Pin AIN1 is electrically coupled to electrode 110.

The system 100 has four branches: a branch that includes electrode 104 and pin CE0, a branch that includes electrode 106 and pin AIN2, a branch that includes electrode 108 and pin AIN3, and a branch that includes electrode 110 and pin AIN1. Two branches are for sensing a first end of the unknown bio-impedance Z_{BODY}, and two other branches are for sensing a second end of the unknown bio-impedance Z_{BODY}. The branch that includes electrode 104 is coupled to the first end of unknown bio-impedance Z_{BODY}. The branch that includes electrode 106 is coupled to the first end of unknown bio-impedance Z_{BODY}. The branch that includes electrode 108 is coupled to the second end of unknown bio-impedance Z_{BODY}. The branch that includes electrode 110 is coupled to the second end of unknown bio-impedance Z_{BODY}. The four branches are connected to respective pins of circuitry 150. Parts of the branches outside of circuitry 150 can represent cables with patches at the end of the cables. Parts of the branches outside of circuitry 150 can also represent conductors or wires having electrodes at the end of the conductors or wires. The conductors and electrodes can be fitted in a wearable device. Optionally, capacitances shown C_{ISO1}, C_{ISO2}, C_{ISO3}, C_{ISO4} can be included between respective pairs of electrodes and pins to provide isolation and protection between the body of the human user and the circuitry within circuitry 150 (e.g., to block DC signals).

Circuitry 150 can include a multiplexer (mux) 112. Mux 112 can be controlled in a manner to connect signal paths of the different pins to different parts of circuitry 150. Mux 112, as used herein, represents a configurable network controllable to connect different parts of circuitry 150 to different pins. For instance, mux 112 can connect different parts of circuitry 150 to different branches connected to the pins (the branches having respective electrodes). Different configurations of mux 112 can form different signal paths or different impedance networks (impedance networks being synonymous with signal paths).

Circuitry 150 can include a signal generator 116 (e.g., sinusoidal signal generator). Signal generator can generate a signal having a peak voltage of V_{PEAK}. The signal generator generates the signal at an output of the signal generator.

Circuitry 150 can include voltage measurement circuitry 118 to measure a voltage across a positive input and a negative input of the voltage measurement circuitry 118. In some embodiments, voltage measurement circuitry 118 can include an instrumentation amplifier (inAmp) 120 with a positive terminal and a negative terminal to sense a voltage difference between the positive terminal and negative terminal, and outputs a voltage output representative of that voltage difference. Voltage measurement circuitry 118 can include a Discrete Fourier Transform (DFT) block 122 and summation block 124 to generate a voltage measurement based on the voltage output from inAmp 120. Components for generating a voltage measurement (e.g., a difference in voltage between two inputs) can differ depending on the implementation.

Circuitry 150 can further include current measurement circuitry 126 to measure a current at an input of the current measurement circuitry 126. In some embodiments, current measurement circuitry 126 can include a transimpedance amplifier (TIA) 128 to convert a current at an input terminal of the TIA 128 to a voltage output representative of the current. Current measurement circuitry 126 can include a DFT block 130 and summation block 132 to generate a current measurement based on the voltage output from TIA 128. Components for generating a current measurement (e.g., an amount of current flowing through an input) can differ depending on the implementation.

To make an impedance measurement, a voltage is generated across the unknown bio-impedance shown as Z_{BODY}. The voltage across the unknown bio-impedance Z_{BODY} can be viewed as V_{A}-V_{B}. The voltage across the unknown bio-impedance Z_{BODY} can be generated or imposed by signal generator 116. Meanwhile, the voltage across the unknown bio-impedance Z_{BODY} is measured by the voltage measurement circuitry 118, and the current through the unknown bio-impedance Z_{BODY} is also measured, by current measurement circuitry 126. The measured voltage and the measured current can be used to derive the impedance value of the unknown bio-impedance Z_{BODY}. Specifically, the impedance value of the unknown bio-impedance Z_{BODY} is related to the voltage measurement divided by the current measurement.

In conventional two-wire impedance measurements, measurement issues can arise from impedances of cables (including contact impedances) being added to the unknown bio-impedance Z_{BODY}, thus corrupting the impedance measurement. For simplicity, the impedances present are lumped together as a contact impedance in each branch. In theory, a four-wire impedance measurement can avoid such issues. When the unknown bio-impedance Z_{BODY} is much higher than the impedances of the cables, the measurements can be sufficiently accurate.

However, in practice, a four-wire impedance measurement can have certain other limitations or non-idealities that can significantly impact the accuracy of the bio-impedance measurement. These limitations can be significant, e.g., when making impedance measurements at low frequencies, high frequencies, certain frequencies, or various frequencies. In some situations, one or more of the contact impedances Z_{E1}, Z_{E2}, Z_{E3}, and Z_{E4} can be greater than the unknown bio-impedance Z_{BODY}. For instance, mechanical and/or environmental reasons (e.g., humidity, movement, hair on skin, etc.) can cause poor contacts, and can severely increase one or more of the contact impedances. In some severe cases, the (magnitude of) contact impedances can be greater than 2 kΩ. In some situations, the optional capacitors C_{ISO1}, C_{ISO2}, C_{ISO3}, C_{ISO4} can also significantly increase or affect the impedances of the cables. In some situations, the contact impedances Z_{E1}, Z_{E2}, Z_{E3}, and Z_{E4} can have an imbalance with each other (e.g., imbalance can be greater than 1 kΩ). These limitations have been found to degrade the accuracy of the four-wire impedance measurement.

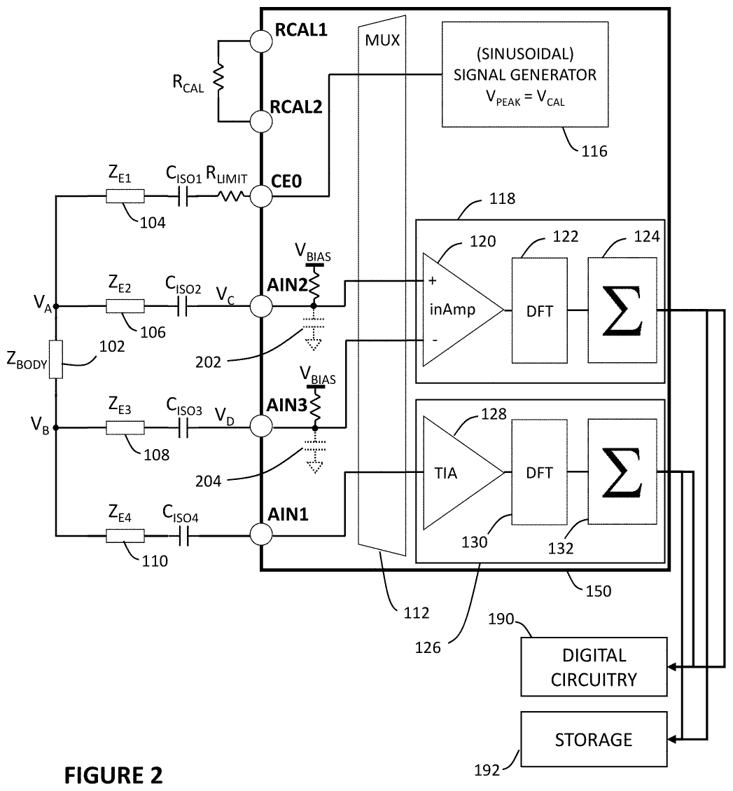

One of the problems causing these limitations that degrade the accuracy of the bio-impedance measurement is that there can be large input capacitances at pin AIN2 and pin AIN3. FIG. 2 illustrates input capacitances present in circuitry that performs a four-wire impedance measurement of bio-impedance, according to some embodiments of the disclosure. Grounded input capacitance 202 can be present at pin AIN2, and grounded input capacitance 204 can also be present at pin AIN3. Grounded input capacitance 202, contact impedance Z_{E2}, and capacitance C_{ISO2} can form a filter. This filter can be problematic because the contact impedance Z_{E2} is unknown, and thus the effect of the filter is unknown as well. Grounded input capacitance 204, contact impedance Z_{E3}, and capacitance C_{ISO3} can also form another filter. This other filter can be problematic because the contact impedance Z_{E3} is unknown, and thus the effect of this other filter is unknown as well. Ideally, voltage V_{A} should be the same as the voltage V_{C}, and voltage V_{B} should be the same as the voltage V_{D}. Due to the grounded input capacitances 202 and 204, at certain frequencies, voltage V_{A} is not the same as the voltage V_{C}, and voltage V_{B} is not the same as the voltage V_{D}. The voltage across V_{A} and V_{B} may not be the same as the voltage across V_{C} and V_{D}. The negative effect of the grounded input capacitances 202 and 204 can be observable at low frequencies and when contact impedances are high, e.g., in the range of hundreds or thousands of Ohms. Furthermore, the grounded input capacitances 202 and 204 can attribute to imbalances in the contact impedances. Imbalances in the contact impedances of the branches can produce different cut-off frequencies, thereby causing different attenuations in each branch.

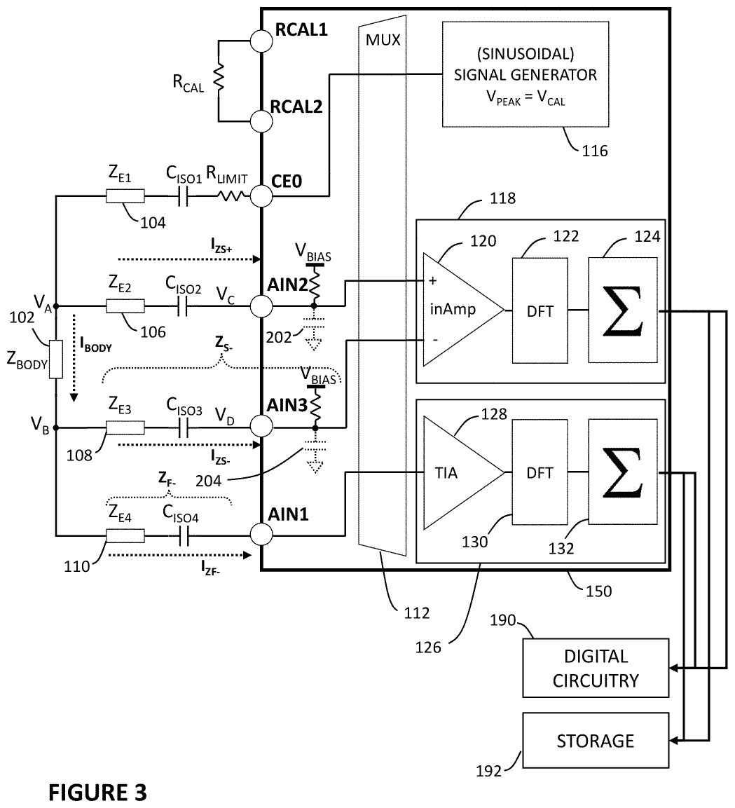

Another problem that may degrade the accuracy of the bio-impedance measurement is current leakage. FIG. 3 illustrates current leakage present in circuitry that performs a four-wire impedance measurement of bio-impedance, according to some embodiments of the disclosure. The current leakage may arise on both of the sense electrodes Zs+ and Zs− due to a finite impedance presented by the parasitic networks at these nodes. As a result, the return current flowing to the TIA 128 through Z_{F-} is not the same as generated by the source. This results in some of the current IBODY flowing through the unknown bio-impedance Z_{BODY} to flow through the branch having electrode 108, and not all of the current IBODY would flow through the branch having electrode 110. In other words, the current I_{ZS-} through the branch having electrode 108 is ideally zero, and the current I_{ZF-} through the branch having electrode 110 is ideally equal to current I_{BODY}. In reality, the current I_{ZS-} is not zero. As a result, the current I_{ZF-} through the branch having electrode 110 is not equal to current I_{BODY}, and part of current I_{BODY} is not measured by the current measurement circuitry 126. The current measurement is corrupted, and thus the impedance measurement is also corrupted. This issue can be exacerbated by high contact impedances in the branches.

Model of Intervening Parasitic Network

A proposed method includes a model of a generalized body network (also referred as the load network) and a generalized intervening parasitic network. The measurement apparatus has a MUX arrangement that can be switched to various configurations and a simple two-wire measurement can be made for each configuration. Six different two-wire measurements are performed to construct a complete Z-matrix of the network as seen from the mux and are calibrated with respect to RCAL. The measurements are taking on known loads (e.g., in the factory) to estimate the intervening parasitic network. The parasitic network is then calibrated and the calibration parameters are stored in flash memory of the system. The calibration parameters are later used to de-embed any measurements taken on unknown loads to estimate the load impedance (Z_{LOAD}) matrix.

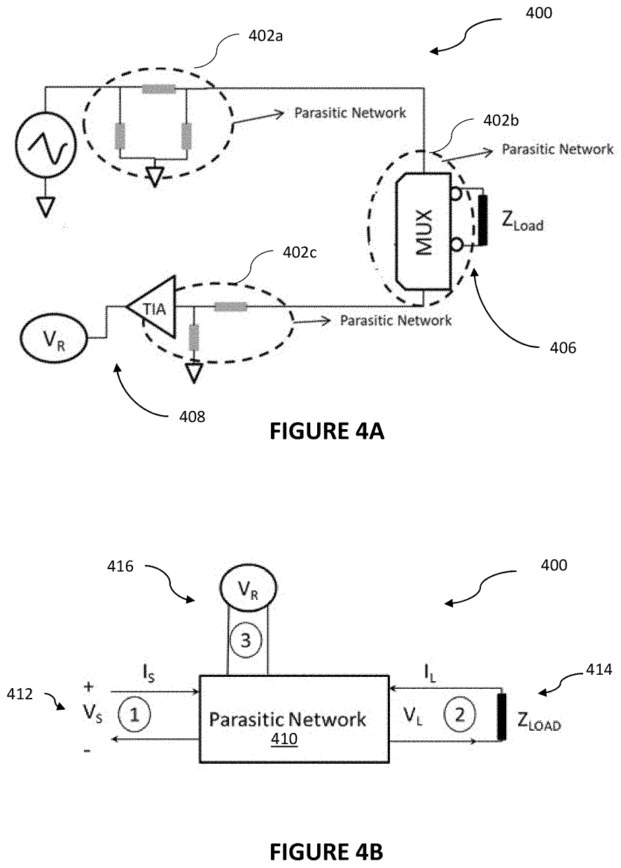

FIGS. 4A and 4B illustrate simplified block diagrams of a method for measuring a simple two-wire impedance with a non-ideal meter. FIG. 4A is a simplified model a system 400, which may be similar or identical to system 100 (FIG. 1). As shown in FIG. 4A, there are three parasitic networks 402a , 402b , 402c , each of which is associated with one of signal generator circuitry 404, a mux 406, and current measurement circuitry 408.

FIG. 4B illustrates a more simplified model of the system 400, in which the parasitic networks 402a , 402b , 402c , are represented as lumped network 410 having three ports 412, 414, 416. Port 412 corresponds to the signal source signal generator circuitry, or signal source, and a voltage V_{S} (measurable quantity) and a current Is. Port 414 corresponds to the load with an inaccessible port voltage V_{L} and an inaccessible port current I_{L}. An ideal voltmeter is connected to Port 416 that measures the port voltage without drawing any port current. In reality, an ideal voltmeter doesn't exist and a real voltmeter is modelled as a combination of an ideal voltmeter and a parasitic network whose effects are absorbed in the lumped network 410.

The voltages may be represented by the following equations:

V_{S}=Z_{SS}*I_{S}+Z_{SL}*I_{L}

V_{L}=Z_{LS}*I_{S}+Z_{LL}*I_{L}

V_{R}=Z_{RS}*I_{S}+Z_{RL}*I_{L}

The load voltage and the load current are also related by the equation

V_{L}=−Z_{LOAD}*I_{L}

Combining the above equations, we get:

ρ_{M}=V_{R}/V_{S}=(a₀+a₁Z_{LOAD})/(1+a₂Z_{LOAD})

where a₀, a₁ and a₂ are complex coefficients per frequency.

Estimation of a₀, a₁ and a₂ occurs through a calibration process that may be run before a measurement session. Three unknowns would need measurements with three different loads. In particular, measurements would need to be made with an open circuit, a short circuit, and R_{CAL}, which may be selected to be approximately twice the expected contact impedance in order to improve accuracy of the system. The complex coefficients per frequency may be saved in a flash memory device of the system The straightforward algebraic equation above would then yield the value of any unknown Z_{LOAD} corresponding to a measured ρ_{M}=V_{R}/V_{S}.

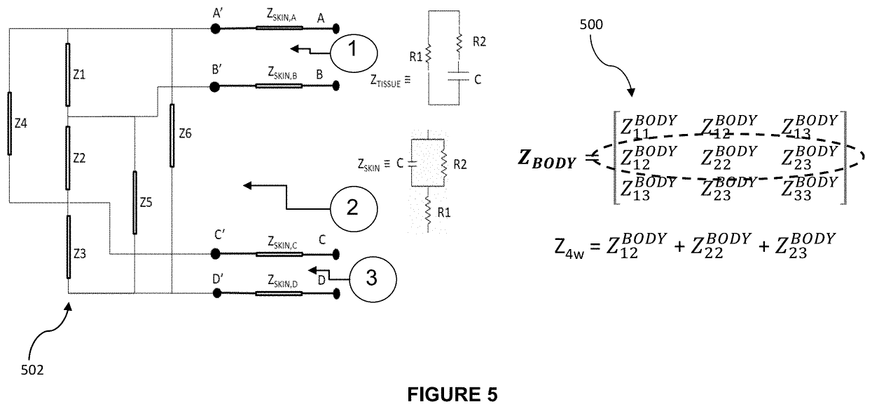

As shown in FIG. 5, the detailed body network may be represented as a 3×3 impedance matrix 500 of a 3-port network 502. Port 1 is defined as the interface between electrodes A and B, port 2 is defined as the interface between electrodes B and C, and port 3 is defined as the interface between electrodes C and D. A and D are defined as force electrodes (F+ and F−) and correspond respectively to electrodes 104 and 110 of FIG. 1, while electrodes B and C are defined as sense electrodes (S+ and S−) and correspond respectively to electrodes 106 and 108 of FIG. 1. An ideal 4-wire impedance may be defined as the sum of the middle row or column of the 3×3 impedance matrix 500, in which Zᵢᵢ^{BODY} is the “looking in” impedance at Port “i” and Zᵢⱼ^{BODY} is the voltage developed on Port “j” due to a 1 A current on Port “i”. It will be recognized that all other ports are open during measurement.

Exemplary Configurations for Measurement Calibration

By configuring mux 112 and making multiple current measurements, it is possible to derive the (unknown) impedances of the system, including the unknown bio-impedance network Z_{BODY} (also referred to interchangeably herein as Z_{LOAD}) and the impedances of the parasitic network, based on a system of equations. The system of equations may be formed through calibration measurements, and several other current measurements of different signal paths formed by configuring mux 112. Mux 112 can selectively couple the output of the signal generator 116 and the input of the current measurement circuitry 126 to different pins (e.g., RCAL1, RCAL2, CE0, AIN2, AIN3, and AIN1). Accordingly, mux 112 can connect the output of the signal generator 116 to the input of the current measurement circuitry 126 through different signal paths, or different impedance networks involving at least some of the unknown impedances. The different signal paths, individually, can include two or more of the unknown impedances of the system: the unknown bio-impedance network Z_{BODY}, and the contact impedances Z_{E1}, Z_{E2}, Z_{E3}, and Z_{E4}. Unique signal paths or unique impedance networks of at least some of the unknown impedances, and the current measurements of the unique signal paths or unique impedance networks, setup a system of equations for the unknown impedances. The unique signal paths or unique impedance networks, together, include each one of the unknown impedances at least once. Each unique signal path or unique impedance network would include at least some of the unknown impedances of the system. Effectively, the signal generator 116 can excite unique signal paths or unique impedance networks formed by mux 112, and the current measurement circuitry 126 can make measurements of current going through the unique signal paths or unique impedance networks.

The current measurements can be performed by the current measurement circuitry 126. The signal processing can be performed in the digital domain, e.g., by digital circuitry 190. Digital circuitry 190 can include specialized digital hardware to perform the signal processing. Digital circuitry 190 can include a microprocessor or microcontroller configured to carry out instructions that implement the signal processing. The digital circuitry 190 can be provided on-chip with circuitry 150 or off-chip (as shown). Digital circuitry 190 can be implemented to control mux 112 to form unique signal paths or unique impedance networks from the signal generator 116 to the current measurement circuitry 126. Computer-readable storage 192 can store the measurements. Computer-readable storage 192 can store the instructions that implement the signal processing. The computer-readable storage 192 can be provided on-chip with circuitry 150 or off-chip (as shown).

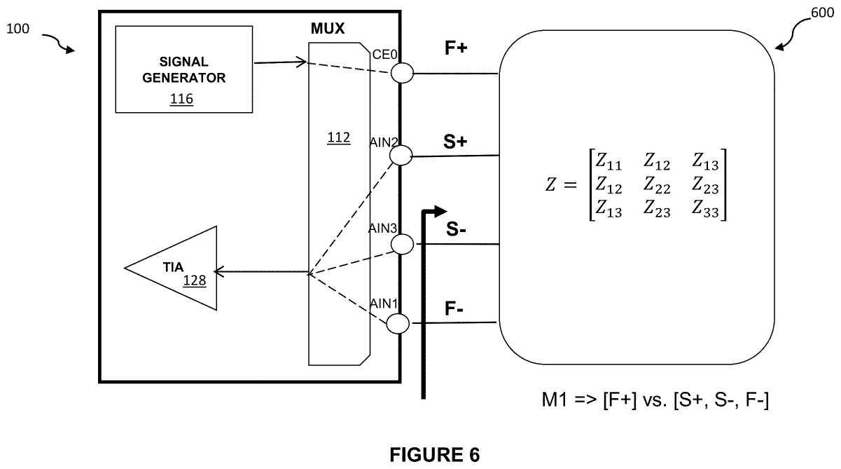

FIGS. 6-11 illustrates calibration measurements, according to some embodiments of the disclosure. The calibration measurement is performed to determine a voltage from the signal generator 116 if it is not already measured or if it is not already known. The system of equations (shown as equations 1-6 below) being formed by the current measurements of unique signal paths going through at least some of the unknown impedances use the voltage measured in the calibration measurement as a numerical constant. The unknown impedances would be derived based further on the voltage measured in the calibration measurement. Determining the voltage from the signal generator 116 can be performed in various ways. An output from the signal generator 116 can be applied to a resistor with a known resistance value, and the current measurement circuitry 126 can measure a current through the resistor. The calibration measurement is represented by: V_{CAL}=I_{CAL}·R_{CAL} (reproduced as equation 1 below). R_{CAL} is a resistor with a known stable resistance value. I_{CAL} is measured by the current measurement circuitry 126. Accordingly, V_{CAL}, which is the voltage from signal generator 116 can be derived.

The resistor with a known resistance value can be provided on-chip with circuitry 150 or off-chip (as shown). The calibration measurement is optional if the peak voltage from the signal generator is known. The calibration measurement may only need to be performed once and does not need to be performed every time impedance measurements are being made.

In the example shown, for the calibration measurement, an (off-chip) resistor R_{CAL} having a known, stable resistance value is coupled across pins RCAL1 and RCAL2. The mux 112 is configured to couple the signal path from pin RCAL1 to the signal generator 116 and to couple the signal path from pin RCAL2 to the current measurement circuitry 126. The mux 112 forms a signal path from the output of signal generator 116 to input of current measurement circuitry 126, and the signal path includes resistor R_{CAL}. The mux 112 connects the output of signal generator 116 to input of current measurement circuitry 126 through the resistor R_{CAL}. The measured current performed by current measurement circuitry is I_{CAL}. With the known resistance value of the resistor R_{CAL}, it is possible to derive the voltage V_{CAL}=I_{CAL}·R_{CAL}. The voltage V_{CAL} represents the (calibrated) voltage from signal generator 116. The measurement of the voltage V_{CAL} across R_{CAL} is determined by measuring a current through R_{CAL}, i.e., through the signal path that includes R_{CAL}, by current measurement circuitry 126.

In accordance with features of embodiments described herein, the value of R_{CAL} may be equal to zero (a short circuit) and infinity (an open circuit), or any other known resistance value in between. In certain embodiments, calibration is performed with R_{CAL} equal to zero, R_{CAL} equal to infinity, and RCAL equal to approximately twice the expected contact impedance to achieve optimal accuracy.

Referring again to FIG. 1, as will be described in greater detail below, by configuring mux 112 and making multiple measurements, it is possible to derive the unknown impedances of the system, which the unknown bio-impedance Z_{BODY}, and the impedance of a parasitic network based on a series of measurements, which may include a calibration measurement, and several current measurements of different, unique signal paths formed by configuring mux 112. Mux 112 can selectively couple the output of the signal generator 116 and the input of the current measurement circuitry 126 to different pins (e.g., RCAL1, RCAL2, CE0, AIN2, AIN3, and AIN1). Accordingly, mux 112 can connect the output of the signal generator 116 to the input of the current measurement circuitry 126 through different signal paths, or different impedance networks involving all of the unknown impedances. The different, unique signal paths, form unique impedance networks, where each unique impedance network combines all of the unknown impedances of the system with a unique topology. Unique signal paths or unique impedance networks each involving all of the unknown impedances, and the current measurements of the unique signal paths or unique impedance networks, setup a system of equations for the unknown impedances. Effectively, the signal generator 116 can excite unique signal paths or unique impedance networks formed by mux 112, and the current measurement circuitry 126 can make measurements of current going through the unique signal paths or unique impedance networks.

Through suitable processing, the current measurements allow the system impedances to be determined. The current measurements can be performed by the current measurement circuitry 126. The signal processing can be performed in the digital domain, e.g., by digital circuitry 190. Digital circuitry 190 can include specialized digital hardware to perform the signal processing. Digital circuitry 190 can include a microprocessor or microcontroller configured to carry out instructions that implement the signal processing. The digital circuitry 190 can be provided on-chip with circuitry 150 or off-chip (as shown). Digital circuitry 190 can be implemented to control mux 112 to form unique signal paths or unique impedance networks from the signal generator 116 to the current measurement circuitry 126. Computer-readable storage 192 can store the measurements. Computer-readable storage 192 can store the instructions that implement the signal processing. The computer-readable storage 192 can be provided on-chip with circuitry 150 or off-chip (as shown).

FIGS. 6-11 are simplified block diagrams of the system 100, including pins CE0, AIN2, AIN3, and AIN1, mux 112, signal generator 116, and TIA 128. FIGS. 6-11 illustrate estimation of six parameters of an impedance matrix 600 using six independent impedance measurements.

In FIG. 6, the mux 112 is configured to couple the signal path from pin CE0 to the output of the signal generator 116, to couple the signal path from pin AIN2 to the input of current measurement circuitry 126, to couple the signal path from pin AIN3 to the input of current measurement circuitry 126, to couple the signal path from pin AIN1 to the input of current measurement circuitry 126. The measured current done by current measurement circuitry 126 is I₁. A matrix M1 encapsulates the relationship between the measured current I₁, measured voltage V_{CAL}, and the unknown impedances in the overall signal path from the signal generator 116 to current measurement circuitry 126 (formed by the mux 112 in the configuration shown in FIG. 6).

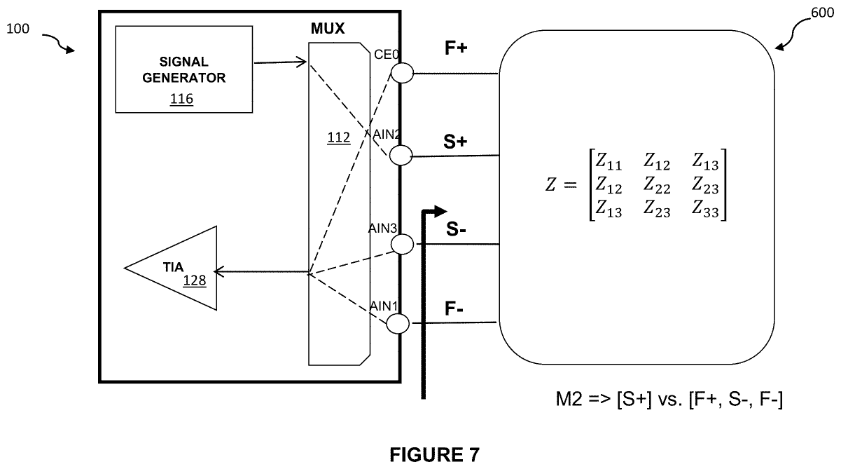

In FIG. 7, the mux 112 is configured to couple the signal path from pin AIN2 to the output of signal generator 116, to couple the signal path from pin CE0 to the input of current measurement circuitry 126, to couple the signal path from pin AIN3 to the input of current measurement circuitry 126, to couple the signal path from pin AIN1 to the input of current measurement circuitry 126. The measured current done by current measurement circuitry 126 is I₂. A matrix M2 encapsulates the relationship between the measured current 12, measured voltage V_{CAL}, and the unknown impedances in the overall signal path from the signal generator 116 to current measurement circuitry 126 (formed by the mux 112 in the configuration shown in FIG. 7).

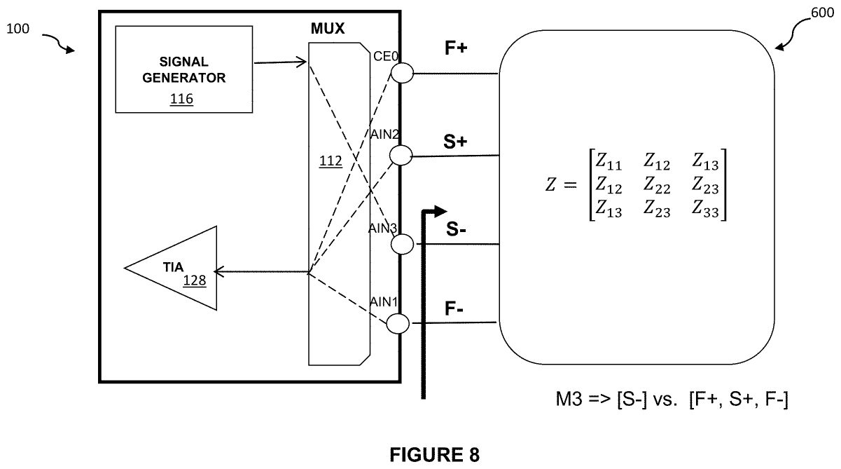

In FIG. 8, the mux 112 is configured to couple the signal path from pin AIN3 to the output of signal generator 116, to couple the signal path from pin CE0 to the input of current measurement circuitry 126, to couple the signal path from pin AIN2 to the input of current measurement circuitry 126, to couple the signal path from pin AIN1 to the input of current measurement circuitry 126. The measured current done by current measurement circuitry 126 is I₃. A matrix M3 encapsulates the relationship between the measured current I₃, measured voltage V_{CAL}, and the unknown impedances in the overall signal path from the signal generator 116 to current measurement circuitry 126 (formed by the mux 112 in the configuration shown in FIG. 8).

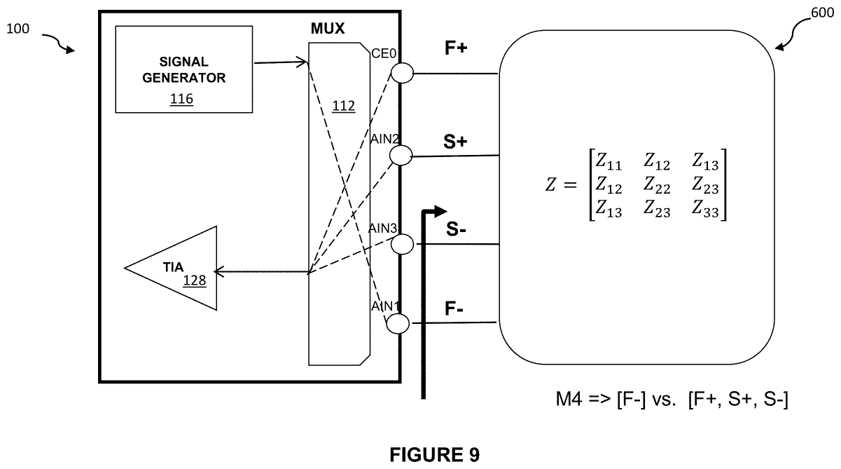

In FIG. 9, the mux 112 is configured to couple the signal path from pin AIN1 to the signal generator 116, to couple the signal path from pin CE0 to the current measurement circuitry 126, to couple the signal path from pin AIN2 to the current measurement circuitry 126, to couple the signal path from pin AIN3 to the current measurement circuitry 126. The measured current done by current measurement circuitry 126 is I₄. A matrix M4 encapsulates the relationship between the measured current I₄, measured voltage V_{CAL}, and the unknown impedances in the overall signal path from the signal generator 116 to current measurement circuitry 126 (formed by the mux 112 in the configuration shown in FIG. 9).

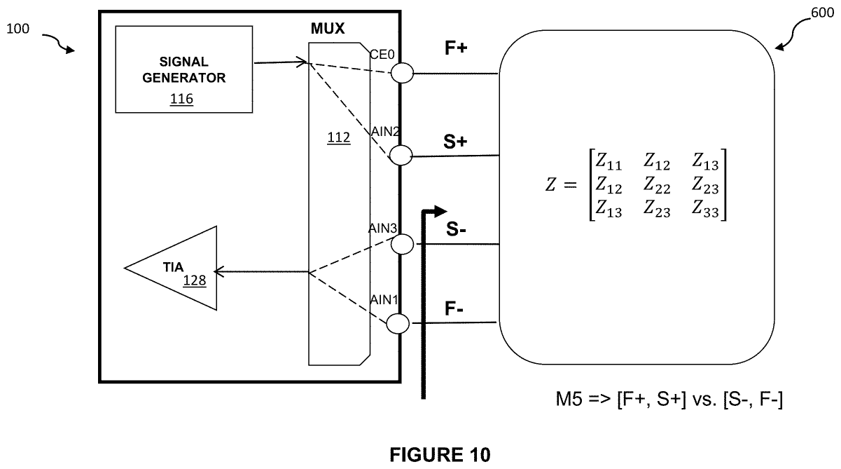

In FIG. 10, the mux 112 is configured to couple the signal path from pin CE0 to the signal generator 116, to couple the signal path from pin AIN2 to the signal generator 116 (as well), to couple the signal path from pin AIN3 to the current measurement circuitry 126, to couple the signal path from pin AIN1 to the current measurement circuitry 126. The measured current done by current measurement circuitry 126 is I₅. A matrix M5 encapsulates the relationship between the measured current I₅, measured voltage V_{CAL}, and the unknown impedances in the overall signal path from the signal generator 116 to current measurement circuitry 126 (formed by the mux 112 in the configuration shown in FIG. 10).

An alternative to the signal path illustrated by FIG. 10 is to connect pin CE0 and pin AIN2 to the input of current measurement circuitry 126, and to connect pin AIN3 and pin AIN1 to the output of signal generator 116.

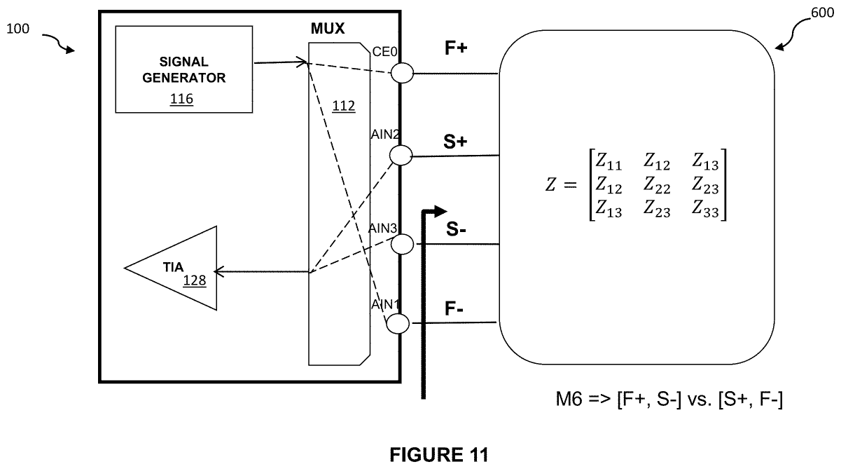

In FIG. 11, the mux 112 is configured to couple the signal path from pin CE0 to the signal generator 116, to couple the signal path from pin AIN2 to the current measurement circuitry 126, to couple the signal path from pin AIN3 to the current measurement circuitry 126, and to couple the signal path from pin AIN1 to the signal generator 116. The measured current done by current measurement circuitry 126 is I₆. Matrix M6 encapsulates the relationship between the measured current I₅, measured voltage V_{CAL}, and the unknown impedances in the overall signal path from the signal generator 116 to current measurement circuitry 126 (formed by the mux 112 in the configuration shown in FIG. 11).

An alternative to the signal path illustrated by FIG. 11 is to connect pin CE0 and pin AIN1 to the input of current measurement circuitry 126, and to connect pin AIN3 and pin AIN2 to the output of signal generator 116.

The measurements shown in FIGS. 6-11 can be performed in any order.

Example Mapping of Impedance Matrix From Mux End to Load End

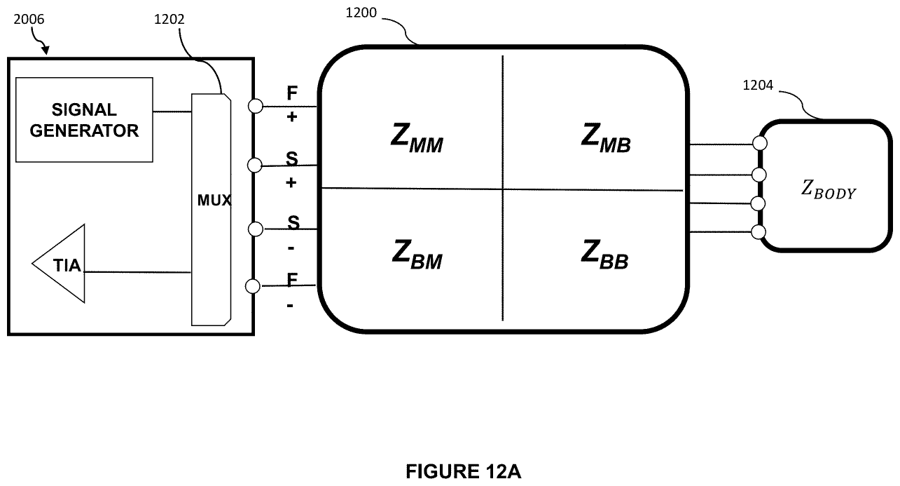

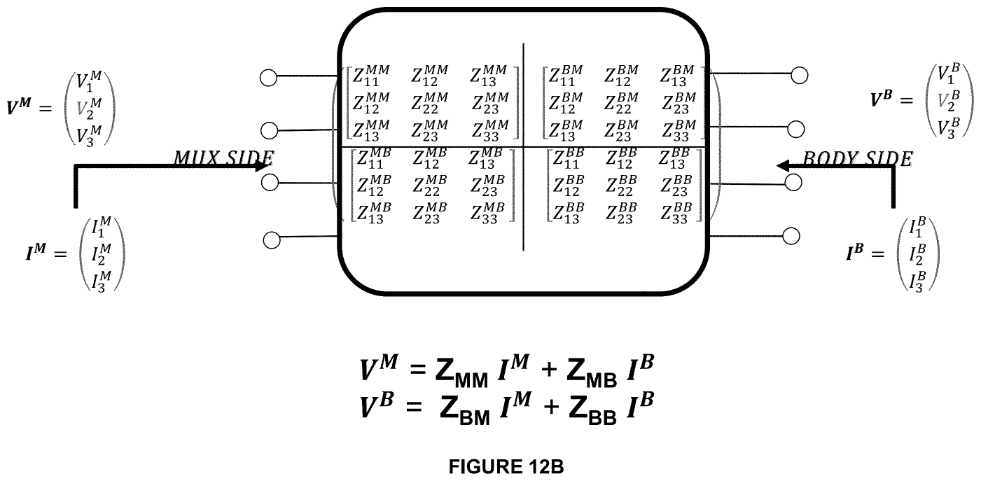

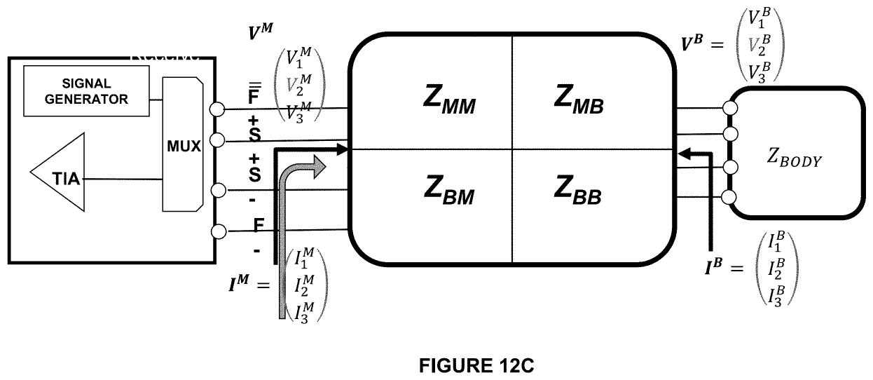

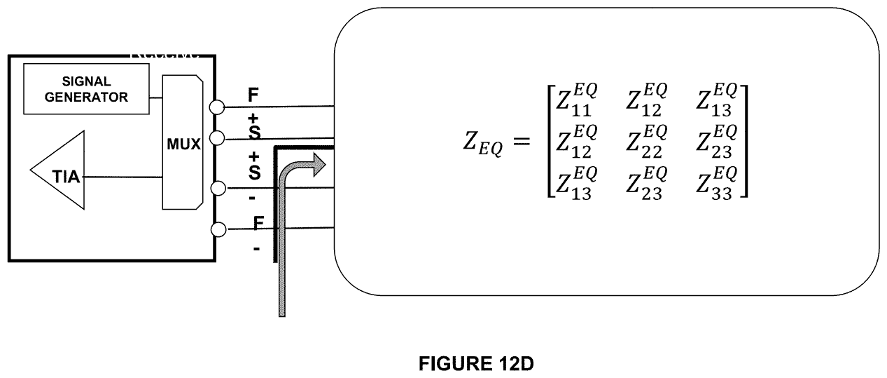

FIGS. 12A-12D illustrate an example mapping of an impedance matrix 1200 representing an intervening (e.g., parasitic) network between a mux end 1202 and load, or body, end 1204 of a system 1206, which in certain embodiments is equivalent to the system 100 (FIG. 1). As previously noted, the parasitic network is an unknown entity between the body network and the on-chip mux of the measurement system. In accordance with features of embodiments described herein, the parasitic network may be modeled as a six-port network, with three mux-side ports and three body-side ports. The impedance matrix 1200 of the parasitic network may be represented as a 6x6 symmetric matrix, including 21 complex unknowns. Impedance matrices Z_{MM} and Z_{BB} are symmetric matrices, whereas impedance matrices Z_{MB} and Z_{BM} are transpose of one another.

As shown in FIGS. 12A-12D, mux-side port voltages (V^{M}) are defined by the equation:

V^{M}=Z_{MM}I^{M}+Z_{MB}I^{B}

And body-side port voltages (V_{B}) are defined by the equation:

V^{B}=Z_{BM}I^{M}+Z_{BB}I^{B}=−Z_{BODY}I^{B}

where I_{M} represents the mux-side port currents and I_{B} represents the body-side port currents. The negative sign indicates the body current flowing out of the body network and into the intervening parasitic network

Using the above equations, V^{M} may further be reduced to:

V^{M}=[Z_{MM}−Z_{MB}(Z_{BODY}+Z_{BB})⁻¹Z_{BM}]^{M}=Z_{EQ}I^{M}

Referring now particularly to FIG. 12D, a “looking in” impedance from the mux 116 may be defined by a matrix 1210 designated Z_{EQ}, where:

Z_{EQ}=Z_{MM}−Z_{MB}(Z_{BODY}+Z_{BB})⁻¹Z_{BM}

It will be recognized that Z_{EQ} comprises a combination of 21 unknown terms of the parasitic network and Z_{BODY}.

Example Calibration Method

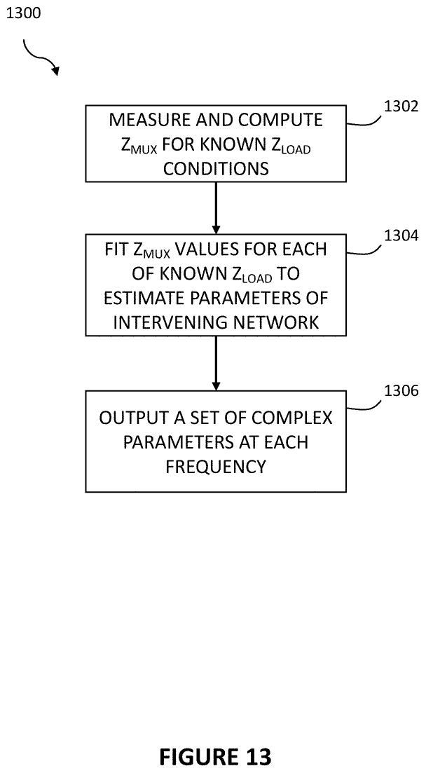

FIG. 13 is a flow diagram illustrating an example calibration method 1300, according to some embodiments of the disclosure. In step 1302, Z_{MUX} (also referred to herein interchangeably as Z_{EQ}) for known Z_{LOAD} conditions is measured and computed as described above. In particular, known load conditions include open, short, and one or more other sequences involving a fixed resistor attached to the load ports in different configurations.

In step 1304, Z_{MUX} values for known Z_{LOAD} conditions are fit to estimate parameters of the intervening (parasitic) network. In a particular embodiment, Levenberg-Marquardt Lsqnonlin( ) is used to perform this operation.

In step 1306, a set of 21 complex parameters for the intervening network at each calibration frequency is output and stored in a flash memory device of the system for later use. In this manner, the intervening network is characterized and may be de-embedded from measurements made using the system to enable estimation of Z_{LOAD} from Z_{MUX}, as described below.

The 21 unknown of the parasitic network embedded in Z_{MUX} (or Z_{EQ}) can be estimated using measurements from multiple known load configurations. Each measurement of Z_{EQ} is a set of six sub-measurements (e.g., as illustrated in FIGS. 6-11). Theoretically, four load configurations should be sufficient to provide enough information to characterize the parasitic network. In practice, however, a few more may be required. An overdetermined set gives better immunity to measurement inaccuracies. A judicious choose of load configurations leads to better estimates of the parasitic parameters.

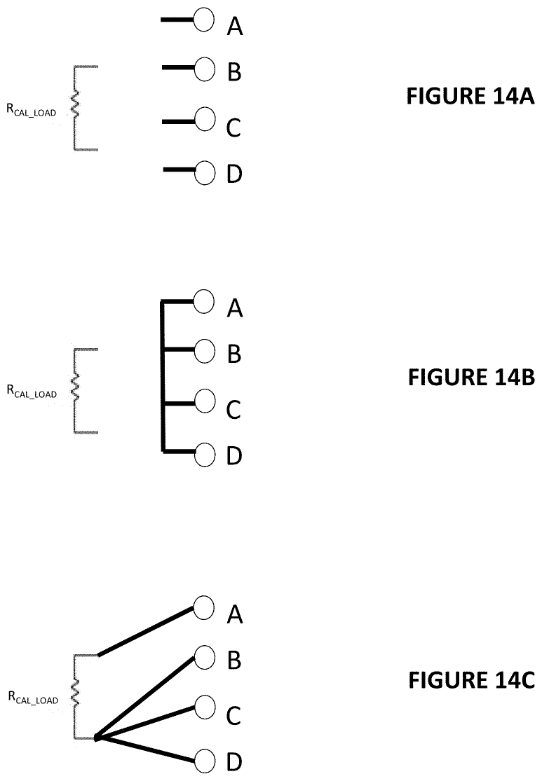

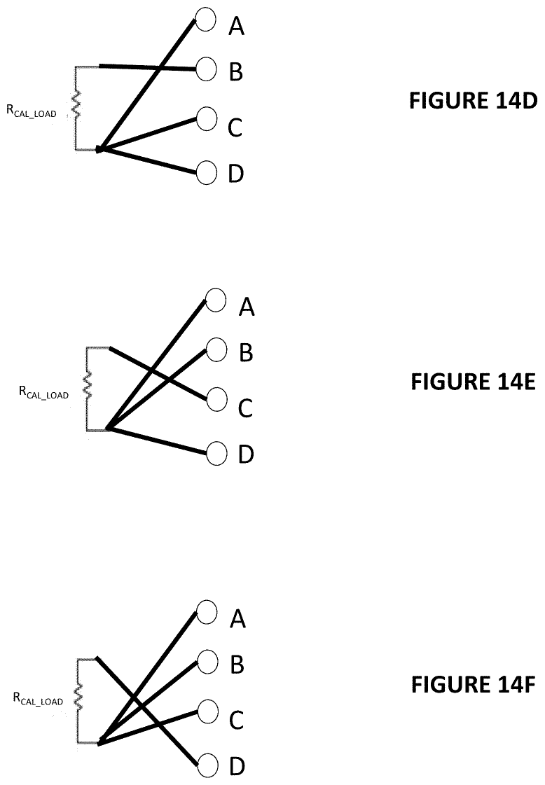

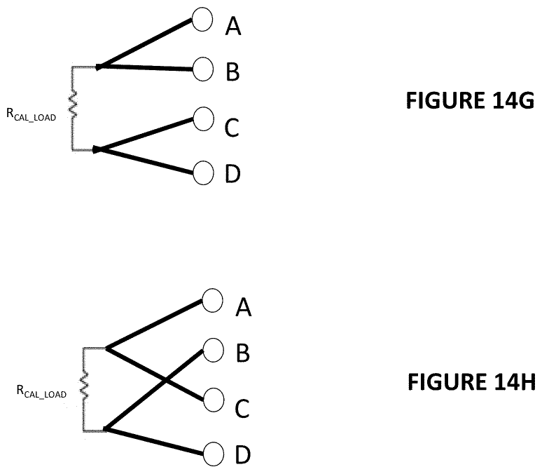

FIGS. 14A-14G illustrate a series of configurations for performing k calibration measurements of Z_{EQ}^{(k)} for k known Z_{BODY}^{(k)}

Z_{EQ}^{(k)}=Z_{MM}−Z_{MB}(Z_{BODY}^{(k)}+Z_{BB})⁻¹Z_{BM}

In particular embodiments, k is equal to eight, resulting in an overdetermined set to solve all 21 unknowns using non-linear fitting tools.

FIG. 14A illustrates measurement of Z_{EQ} where Z_{BODY} is an open circuit.

FIG. 14B illustrates measurement of Z_{EQ} where Z_{BODY} is a short circuit.

FIG. 14C illustrates measurement of Z_{EQ} where Z_{BODY} is R_{CAL_LOAD} connected between [A] vs. [B,C,D] where nodes B,C and D are shorted together.

FIG. 14D illustrates measurement of Z_{EQ} where Z_{BODY} is R_{CAL_LOAD} connected between [B] vs. [A,C,D] where nodes A,C and D are shorted together.

FIG. 14E illustrates measurement of Z_{EQ} where Z_{BODY} is R_{CAL_LOAD} connected between [C] vs. [A,B,D] where nodes A,B and D are shorted together.

FIG. 14F illustrates measurement of Z_{EQ} where Z_{BODY} is R_{CAL_LOAD} connected between [D] vs. [A,B,C] where nodes A,B and C are shorted together.

FIG. 14G illustrates measurement of Z_{EQ} where Z_{BODY} is R_{CAL_LOAD} connected between [A,B] vs. [C,D] where nodes A and B are shorted together and nodes C and D are shorted together.

FIG. 14H illustrates measurement of Z_{EQ} where Z_{BODY} is R_{CAL_LOAD} connected between [A,C] vs. [B,D] where nodes A and C are shorted together and nodes B and D are shorted together.

These parameters are then saved in a non-volatile memory (e.g., a flash memory device or, a EEPROM) and used later to de-embed an unknown load Z_{LOAD}.

Example De-Embedding Method

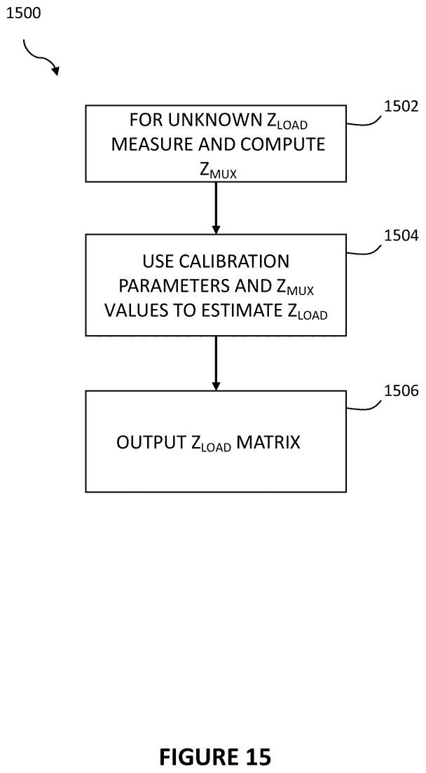

FIG. 15 is a flow diagram illustrating an example de-embedding method 1500, according to some embodiments of the disclosure. In step 1502, for an unknown load, Z_{MUX} is measured and computed as described above.

In step 1504, the calibration parameters output in step 1306 (FIG. 13) are accessed (e.g., from the flash memory device) and used to estimate Z_{LOAD} from the Z_{MUX} values measured and computed in step 1502. In a particular embodiment, Levenberg-Marquardt Lsqnonlin( ) is used to perform this operation.

In step 1506, the estimated Z_{LOAD} is output. The ideal four-wire impedance (bio-impedance) may be obtained by adding the center row (or column) of the Z_{LOAD}. The (1,1), (1,2), (2,3), and (3,3) terms of Z_{LOAD} are indicators of the contact impedances (four branch impedances), respectively, and may be derived using digital circuitry 192 (FIG. 1).

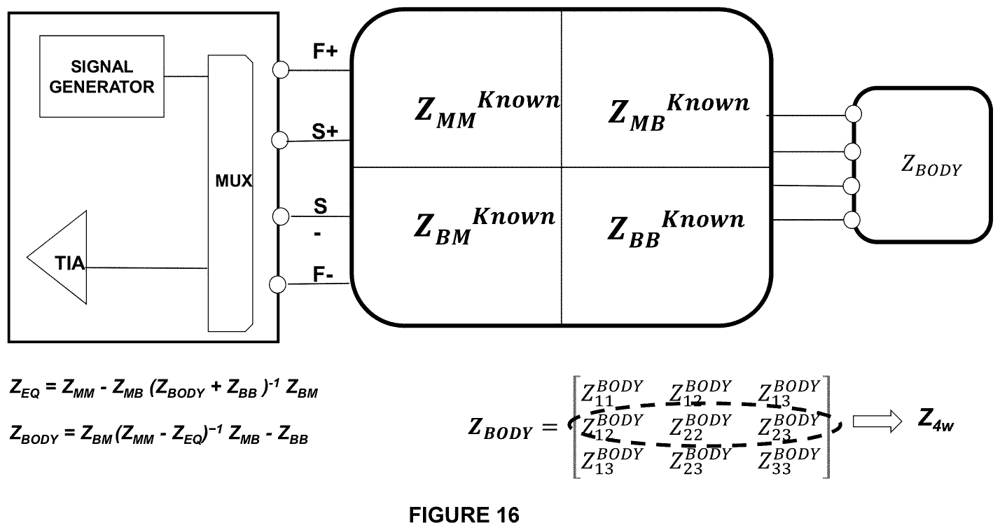

FIG. 16 is a block diagram illustrating the de-embedding process described with reference to FIG. 15.

Example Data Processing System for Use in Implementing Particular Embodiments

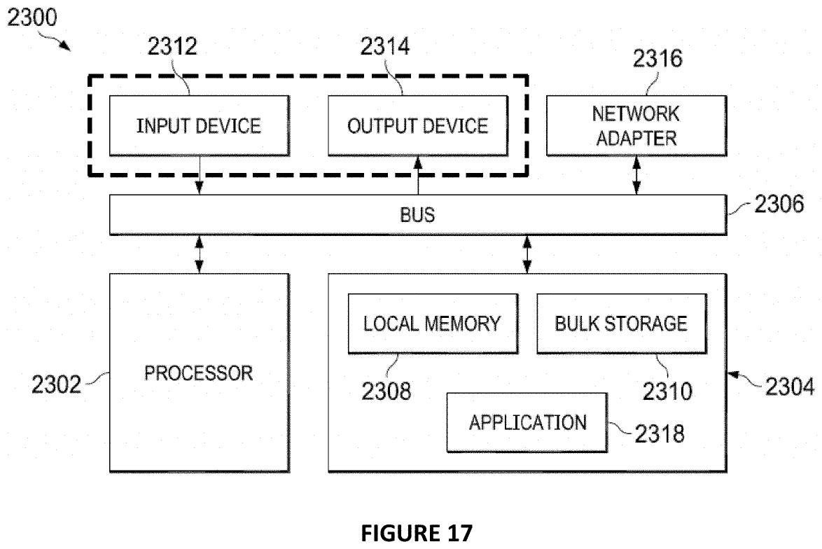

FIG. 17 provides a block diagram illustrating an example processing system 2300 that may be configured to perform one or more of the operations described above according to some embodiments of the present disclosure.

As shown in FIG. 17, the processing system 2300 may include at least one processor 2302, e.g., a hardware processor 2302, coupled to memory elements 2304 through a system bus 2306. As such, the processing system may store program code within memory elements 2304. Further, the processor 2302 may execute the program code accessed from the memory elements 2304 via a system bus 2306. In one aspect, the processing system may be implemented as a computer that is suitable for storing and/or executing program code. It should be appreciated, however, that the processing system 2300 may be implemented in the form of any system including a processor and a memory that is capable of performing the functions described within this disclosure.

In some embodiments, the processor 2302 can execute software or an algorithm to perform the activities as discussed in the present disclosure. The processor 2302 may include any combination of hardware, software, or firmware providing programmable logic, including by way of non-limiting example a microprocessor, a digital signal processor (DSP), a field-programmable gate array (FPGA), a programmable logic array (PLA), an application specific IC (ASIC), or a virtual machine processor. The processor 2302 may be communicatively coupled to the memory element 2304, for example in a direct-memory access (DMA) configuration, so that the processor 2302 may read from or write to the memory elements 2304.

In general, the memory elements 2304 may include any suitable volatile or non-volatile memory technology, including double data rate (DDR) random access memory (RAM), synchronous RAM (SRAM), dynamic RAM (DRAM), flash, read-only memory (ROM), optical media, virtual memory regions, magnetic or tape memory, or any other suitable technology. Unless specified otherwise, any of the memory elements discussed herein should be construed as being encompassed within the broad term “memory.” The information being measured, processed, tracked or sent to or from any of the components of the processing system 2300 could be provided in any database, register, control list, cache, or storage structure, all of which can be referenced at any suitable timeframe. Any such storage options may be included within the broad term “memory” as used herein. Similarly, any of the potential processing elements, modules, and machines described herein should be construed as being encompassed within the broad term “processor.” Each of the elements shown in the present figures can also include suitable interfaces for receiving, transmitting, and/or otherwise communicating data or information in a network environment so that they can communicate with, e.g., the processing system 2300.

In certain example implementations, portions of mechanisms described herein may be implemented by logic encoded in one or more tangible media, which may be inclusive of non-transitory media, e.g., embedded logic provided in an ASIC, in DSP instructions, software (potentially inclusive of object code and source code) to be executed by a processor, or other similar machine, etc. In some of these instances, memory elements, such as e.g., the memory elements 2304 shown in FIG. 17, can store data or information used for the operations described herein. This includes the memory elements being able to store software, logic, code, or processor instructions that are executed to carry out the activities described herein. A processor can execute any type of instructions associated with the data or information to achieve the operations detailed herein. In one example, the processors, such as e.g., the processor 2302 shown in FIG. 17, could transform an element or an article (e.g., data) from one state or thing to another state or thing. In another example, the activities outlined herein may be implemented with fixed logic or programmable logic (e.g., software/computer instructions executed by a processor) and the elements identified herein could be some type of a programmable processor, programmable digital logic (e.g., an FPGA, a DSP, an erasable programmable read-only memory (EPROM), an electrically erasable programmable read-only memory (EEPROM)) or an ASIC that includes digital logic, software, code, electronic instructions, or any suitable combination thereof.

The memory elements 2304 may include one or more physical memory devices such as, for example, local memory 2308 and one or more bulk storage devices 2310. The local memory may refer to RAM or other non-persistent memory device(s) generally used during actual execution of the program code. A bulk storage device may be implemented as a hard drive or other persistent data storage device. The processing system 2300 may also include one or more cache memories (not shown) that provide temporary storage of at least some program code in order to reduce the number of times program code must be retrieved from the bulk storage device 2310 during execution.

As shown in FIG. 17, the memory elements 2304 may store an application 2318. In various embodiments, the application 2318 may be stored in the local memory 2308, the one or more bulk storage devices 2310, or apart from the local memory and the bulk storage devices. It should be appreciated that the processing system 2300 may further execute an operating system (not shown in FIG. 17) that can facilitate execution of the application 2318. The application 2318, being implemented in the form of executable program code, can be executed by the processing system 2300, e.g., by the processor 2302. Responsive to executing the application, the processing system 2300 may be configured to perform one or more operations or method steps described herein.

Input/output (I/O) devices depicted as an input device 2312 and an output device 2314, optionally, can be coupled to the processing system. Examples of input devices may include, but are not limited to, a keyboard, a pointing device such as a mouse, or the like. Examples of output devices may include, but are not limited to, a monitor or a display, speakers, or the like. In some embodiments, the output device 2314 may be any type of screen display, such as plasma display, liquid crystal display (LCD), organic light emitting diode (OLED) display, electroluminescent (EL) display, or any other indicator, such as a dial, barometer, or LEDs. In some implementations, the system may include a driver (not shown) for the output device 2314. Input and/or output devices 2312, 2314 may be coupled to the processing system either directly or through intervening I/O controllers.

In an embodiment, the input and the output devices may be implemented as a combined input/output device (illustrated in FIG. 17 with a dashed line surrounding the input device 2312 and the output device 2314). An example of such a combined device is a touch sensitive display, also sometimes referred to as a “touch screen display” or simply “touch screen.” In such an embodiment, input to the device may be provided by a movement of a physical object, such as e.g., a stylus or a finger of a user, on or near the touch screen display.

A network adapter 2316 may also, optionally, be coupled to the processing system to enable it to become coupled to other systems, computer systems, remote network devices, and/or remote storage devices through intervening private or public networks. The network adapter may comprise a data receiver for receiving data that is transmitted by said systems, devices and/or networks to the processing system 2300, and a data transmitter for transmitting data from the processing system 2300 to said systems, devices and/or networks. Modems, cable modems, and Ethernet cards are examples of different types of network adapter that may be used with the processing system 2300.

### Select Examples

Example 1 is a method for estimating an impedance matrix of parasitic network disposed between a first network and a second network of a bio-impedance measurement system, the method comprising determining an impedance matrix for the first network (Z_{MUX}) based on an impedance matrix for the second network (Z_{LOAD}) for at least one known load condition; and fitting Z_{MUX} values for Z_{LOAD} for the at least one known load condition to estimate parameters of the impedance matrix of the intervening network.

Example 2 provides the method of example 1, wherein the each of first network, the second network and the intervening parasitic network is a multiport networks with at least two ports.

Example 3 provides the method of example 1, wherein the fitting is performed using a non-linear optimization algorithm.

Example 4 provides the method of example 2, where the optimization algorithm used is a Levenberg-Marquardt algorithm.

Example 5 provides the method of example 1, further comprising storing the estimated intervening network impedance matrix parameters.

Example 6 provides the method of example 5, wherein the estimated intervening network impedance matrix parameters are stored in a flash memory device of the bio-impedance measurement system.

Example 7 provides the method of example 5, further comprising subsequent to the storing, determining the Zmux for an unknown load; and estimating the Z_{LOAD} for the unknown load using the stored estimated intervening network impedance matrix parameters and the determined Zmux .

Example 8 provides the method of example 7, wherein the estimating the Z_{LOAD} for the unknown load is performed using a Levenberg-Marquardt algorithm.

Example 9 provides the method of example 7, further comprising determining the bio-impedance and contact impedances from the estimated Z_{LOAD} for the unknown load.

Example 10 provides the method of example 1, wherein the known load condition comprises a load condition selected from the group consisting of a short circuit, an open circuit, and twice an expected contract impedance of the bio-impedance measurement system.

Example 11 is a method for estimating an impedance matrix of an unknown load connected to a bio-impedance measurement system including a multiplexer (mux) network and a parasitic impedance network disposed between the mux impedance network, the method comprising determining an impedance matrix for the mux network (Z_{MUX}) for the unknown load; and estimating an impedance matrix for the unknown load (Z_{LOAD}) using a stored estimated intervening network impedance matrix parameters and the determined Z_{MUX}.

Example 12 provides the method of example 11, wherein the estimating the Z_{LOAD} for the unknown load is performed using a Levenberg-Marquardt algorithm.

Example 13 provides the method of example 11, further comprising determining the bio-impedance and contact impedances from the estimated Z_{LOAD} for the unknown load.

Example 14 provides the method of example 11, further comprising, prior to the determining an impedance matrix for the mux network (Z_{MUX}) for the unknown load determining the Z_{MUX} based on the Z_{LOAD} for at least one known load condition; and fitting Zmuxvalues for Z_{LOAD} for the at least one known load condition to estimate the parameters of the impedance matrix of the intervening network.

Example 15 provides the method of example 14, wherein the fitting is performed using a Levenberg-Marquardt algorithm.

Example 16 provides the method of example 14, further comprising storing the estimated intervening network impedance matrix parameters in a flash memory device of the bio-impedance measurement system.

Example 17 provides the method of example 11, wherein the known load condition comprises at least one of a short circuit, an open circuit, and a resistance value that is twice an expected contract impedance of the bio-impedance measurement system.

Example 18 provides an apparatus for estimating an impedance matrix of parasitic network disposed between a first network and a second network of a bio-impedance measurement system, the apparatus comprising circuitry for determining an impedance matrix for the first network (Z_{MUX}) based on an impedance matrix for the second network (Z_{LOAD}) for at least one known load condition; circuitry for fitting Z_{MUX} values for Z_{LOAD} for the at least one known load condition to estimate parameters of the impedance matrix of the intervening network; and a memory device for storing the estimated intervening network impedance matrix parameters.

Example 19 provides the apparatus of example 18, further comprising circuitry for determining the Z_{MUX} for an unknown load; and circuitry for estimating the Z_{LOAD} for the unknown load using the stored estimated intervening network impedance matrix parameters and the determined Z_{MUX}.

Example 20 provides the apparatus of example 19, wherein the known load condition comprises at least one of a short circuit, an open circuit, and an impedance value equal to two times an expected contract impedance of the bio-impedance measurement system.

Variations and Implementations

The unique signal paths illustrated by the disclosure are not meant to be limiting. Other topologies, schemes for exciting and measuring the signal paths can be implemented and are envisioned by the disclosure.

Moreover, certain embodiments discussed above can be provisioned in digital signal processing technologies for medical imaging, patient monitoring, medical instrumentation, and home healthcare. The embodiments herein can also be beneficial to other applications requiring an accurate impedance measurement using at least four electrodes.

In the discussions of the embodiments above, various electrical components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure.

Parts of various circuitry for deriving unknown impedances can include electronic circuitry to perform the functions described herein. In some cases, one or more parts of the circuitry can be provided by a processor specially configured for carrying out the functions described herein. For instance, the processor may include one or more application specific components, or may include programmable logic gates which are configured to carry out the functions describe herein. The circuitry can operate in analog domain, digital domain, or in a mixed signal domain. In some instances, the processor may be configured to carrying out the functions described herein by executing one or more instructions stored on a non-transitory computer medium. In some embodiments, an apparatus can include means for performing or implementing one or more of the functionalities describe herein.

It is also imperative to note that all of the specifications, dimensions, and relationships outlined herein (e.g., the number of processors, logic operations, etc.) have only been offered for purposes of example and teaching only. Such information may be varied considerably without departing from the spirit of the present disclosure. The specifications apply only to one non-limiting example and, accordingly, they should be construed as such. In the foregoing description, example embodiments have been described with reference to particular processor and/or component arrangements. Various modifications and changes may be made to such embodiments without departing from the scope of the disclosure. The description and drawings are, accordingly, to be regarded in an illustrative rather than in a restrictive sense.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this specification. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in this specification, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It is also important to note that the functions related to deriving unknown impedances, illustrate only some of the possible functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure.

What is claimed is:

1. A method for estimating an impedance matrix of parasitic network disposed between a first network and a second network of a bio-impedance measurement system, the method comprising:
determining an impedance matrix for the first network (Z_{MUX}) based on an impedance matrix for the second network (Z_{LOAD}) for at least one known load condition;
fitting Z_{MUX} values for Z_{LOAD} for the at least one known load condition to estimate parameters of the impedance matrix of the intervening network.

2. The method of claim 1, wherein the each of first network, the second network and the intervening parasitic network is a multiport networks with at least two ports.

3. The method of claim 1, wherein the fitting is performed using a non-linear optimization algorithm.

4. The method of claim 2, where the optimization algorithm used is a Levenberg-Marquardt algorithm.

5. The method of claim 1, further comprising storing the estimated intervening network impedance matrix parameters.

6. The method of claim 5, wherein the estimated intervening network impedance matrix parameters are stored in a flash memory device of the bio-impedance measurement system.

7. The method of claim 5, further comprising:
subsequent to the storing, determining the Z_{MUX} for an unknown load; and
estimating the Z_{LOAD} for the unknown load using the stored estimated intervening network impedance matrix parameters and the determined Z_{MUX}.

8. The method of claim 7, wherein the estimating the Z_{LOAD} for the unknown load is performed using a Levenberg-Marquardt algorithm.

9. The method of claim 7, further comprising determining the bio-impedance and contact impedances from the estimated Z_{LOAD} for the unknown load.

10. The method of claim 1, wherein the known load condition comprises a load condition selected from the group consisting of a short circuit, an open circuit, and twice an expected contract impedance of the bio-impedance measurement system.

11. A method for estimating an impedance matrix of an unknown load connected to a bio-impedance measurement system including a multiplexer (mux) network and a parasitic impedance network disposed between the mux impedance network, the method comprising:
determining an impedance matrix for the mux network (Z_{MUX}) for the unknown load; and
estimating an impedance matrix for the unknown load (Z_{LOAD}) using a stored estimated intervening network impedance matrix parameters and the determined Z_{MUX}.

12. The method of claim 11, wherein the estimating the Z_{LOAD} for the unknown load is performed using a Levenberg-Marquardt algorithm.

13. The method of claim 11, further comprising determining the bio-impedance and contact impedances from the estimated Z_{LOAD} for the unknown load.

14. The method of claim 11, further comprising, prior to the determining an impedance matrix for the mux network (Z_{MUX}) for the unknown load:
determining the Z_{MUX} based on the Z_{LOAD} for at least one known load condition; and
fitting Z_{MUX} values for Z_{LOAD} for the at least one known load condition to estimate the parameters of the impedance matrix of the intervening network.

15. The method of claim 14, wherein the fitting is performed using a Levenberg-Marquardt algorithm.

16. The method of claim 14, further comprising storing the estimated intervening network impedance matrix parameters in a flash memory device of the bio-impedance measurement system.

17. The method of claim 11, wherein the known load condition comprises at least one of a short circuit, an open circuit, and a resistance value that is twice an expected contract impedance of the bio-impedance measurement system.

18. Apparatus for estimating an impedance matrix of parasitic network disposed between a first network and a second network of a bio-impedance measurement system, the apparatus comprising:
circuitry for determining an impedance matrix for the first network (Z_{MUX}) based on an impedance matrix for the second network (Z_{LOAD}) for at least one known load condition;
circuitry for fitting Z_{MUX} values for Z_{LOAD} for the at least one known load condition to estimate parameters of the impedance matrix of the intervening network; and
a memory device for storing the estimated intervening network impedance matrix parameters.

19. The apparatus of claim 18, further comprising:
circuitry for determining the Z_{MUX} for an unknown load; and
circuitry for estimating the Z_{LOAD} for the unknown load using the stored estimated intervening network impedance matrix parameters and the determined Z_{MUX}.

20. The apparatus of claim 19, wherein the known load condition comprises at least one of a short circuit, an open circuit, and an impedance value equal to two times an expected contract impedance of the bio-impedance measurement system.
